(19) United States
(12) Patent Application Publication
(10) Pub. No.: US 2012/0058560 A1
(43) Pub. Date: Mar. 8, 2012

(54) Bioreactor System

(76) Inventors: Christian Derichs, Aachen (DE); Ulrike Koropp, Aachen (DE); Jan Hansmann, Stuttgart (DE); Michaela Kaufmann, Schwelm (DE); Heike Walles, Sindelfingen (DE)

(21) Appl. No.: 13/320,254

(22) PCT Filed: Feb. 20, 2010
(86) PCT No.: PCT/EP10/01081

§ 371 Date: Nov. 11, 2011

(30) Foreign Application Priority Data
May 15, 2009 (DE) 10 2009 022 354.1

(51) Int. Cl. C12N 5/02; C12M 1/12

(52) U.S. Cl. 435/401; 435/297.1; 435/287.1

(57) ABSTRACT

Bioreactors may be used for the cultivation of cells, in particular of adherent cells, and, in particular for the cultivation and propagation of cell cultures, and utilized in methods for the cultivation of cells. A particular area of application is the use of the bioreactors in the GMP-compliant, fully automatic cultivation and propagation of cells.

### BACKGROUND

The invention relates to bioreactors for the cultivation of cells, in particular of adherent cells, to the use of the bioreactors, in particular for the cultivation and propagation of cell cultures, and to methods for the cultivation of cells using the bioreactors according to the invention. A particular area of application is the use of the bioreactors in the GMP-compliant, fully automatic cultivation and propagation of cells.

In the technical field of tissue engineering, particularly in relation to regenerative medicine, there is the need to automate in a GMP-compliant manner biological laboratory processes under clean room conditions. A higher yield, higher process safety and also standardisable process optimisation and process control are to be achieved in this way.

Certain standards in laboratory-scale production have become established over the years. Disposable articles made of injection-moulded polypropylene or polystyrene are thus frequently used, as the very cost-intensive cleaning and disinfection of the sample containers is in this way dispensed with. The cells required for building up the tissue constructs are firstly isolated from a biopsate and then cultivated in different sized cell culture dishes, flasks or multiwell plates over a period of several days.

For the purposes of cultivation, the isolated primary cells are firstly resuspended in specific cell culture media. The cell suspension is subsequently pipetted into disposable culture vessels in which the cells adhere in an undefined manner to the specially pretreated plastic surfaces. After an incubation time of several days in an incubator and regularly exchanging the old cell culture medium with fresh cell culture medium, the cell culture, which has grown with sufficient density, i.e. which is confluent, is detached from the plastics material surface of the cell culture vessels. The detaching process is carried out either purely enzymatically, for example using a trypsin/EDTA-containing solution, or in conjunction with mechanical excitation. In this case, both the enzyme activity and the mechanical stress have negative effects on the vitality of the cells.

The conventional cell culture vessels are inadequate for automated production in relatively large quantities. Cells proliferate only when specific cell-typical conditions are met. An important factor in this regard is for example the seeding density, i.e. the cell concentration at which the isolated cells are introduced into a culture vessel. On the one hand, the seeding density must be selected so as to be sufficiently high to enable the cells to build up the mutual cell/cell contacts necessary for proliferation; on the other hand, the seeding density must be low enough to provide sufficient growth area. In manual laboratory operation, multiple passaging, i.e. a detaching of the cells and the cultivation in a new, larger vessel, ensures that these conditions are met. The process of subcultivation includes a large number of manual operations, such as the addition and removal of solutions, incubation in an incubator, microscopic monitoring of cell detachment, supporting mechanical action, transferring the cell suspension to a centrifuge tube, cell count, centrifugation, removing the supernatant, resuspending of cells in fresh medium and renewed seeding. This leads to increased consumption of pipettes and disposable vessels. When carrying out the individual operations, laboratory staff often apply different handling techniques and make individual decisions. This concerns for example the length of the enzyme reaction, the monitoring of the cell detachment, the selection as to whether, and if so what, mechanical support is applied, or the selection of the vessel for transferring the cell culture. The growth behaviour and the propagation speed of different cell types are also to be taken into account in the cultivation thereof. Overall, the process of unitary cultivation therefore places high demands on an automated system.

Previous attempted solutions for automated cell culture systems have restricted themselves to copying the sequence of the manual laboratory process by using adapted culture vessels. Thus, for example, the CELLSTAR® AutoFlask™ cell culture flask from the company Greiner Bio-One is adapted in its geometry and in its handling to use in existing automated systems. In this case, the cells are passaged in the same manner as in conventional manual processes.

The method of cultivating cells on PET membranes is used as standard in the laboratory. For this purpose, use is made of cell culture inserts, such as they are known for example from WO 2004/020571 A2. Nevertheless, these are available only in a limited size. The use of insert membranes has in the manual laboratory process the draw-back that the growth of the cell culture during the culturing period cannot be monitored under a microscope.

### SUMMARY OF THE INVENTION(S)

The invention is based on the technical problem of providing devices and methods allowing an improved and/or simplified cultivation and expansion of cells over the prior art.

The invention is also based on the technical problem of providing methods and devices allowing a cultivation and expansion of cells that spares the cells.

The invention is also based on the technical problem of providing methods and devices allowing an automated cultivation and expansion of cells.

The invention is also based on the technical problem of providing devices and methods allowing a simple cultivation and expansion of cells.

The invention is also based on the technical problem of providing devices and methods which avoid damage to cells as a result of multiple passaging, while in particular at the same time the culturing period and growth time are extended for the first passage.

The invention was also based on the technical problem of providing methods and devices allowing cells to be detached from the cultivation surface in a manner that spares the adherent cells.

The technical problem underlying the invention is solved by the subject matters of the independent patent claims.

In particular, the technical problem underlying the invention is solved by a bioreactor according to claim 1.

In particular, the technical problem underlying the invention is solved by a bioreactor for the cultivation of cells, wherein the reactor space of the bioreactor is subdivided by a membrane unit which is liquid-permeable, at least in partial regions, into two reactor regions and wherein each of these two reactor regions has an opening suitable for letting in and/or letting out a liquid.

The invention therefore relates to a bioreactor with an integrated cell growth membrane. The bioreactor surrounds, as the housing, a reactor space, also referred to as the reactor chamber. This reactor space is split up by the membrane unit, which is liquid-permeable in partial regions, into two reactor regions. The bioreactor therefore has two reactor regions or reactor space regions.

According to the invention, the bioreactor is preferably used for the cultivation of adherent cells.

In relation to the present invention, the term “adherent cells” refers to cells of the type which can be cultured in cell culture medium on an inert surface, for example a reaction vessel, as a monolayer or as a multilayer, but in particular as a monolayer. The adherent cells contact the surface and adhere thereto. Adherent cells usually form a continuous cell layer. Adherent cells often display a density-dependent proliferation inhibition, also referred to as contact inhibition, which occurs in particular when the confluence is exceeded. Adherent cells often derive from tissues, such as skin, muscles, nerves, the liver, kidneys. Examples of adherent cells are fibroblasts, HeLa cells and many tumour cells. The media which are known to the person skilled in the art and are selected depending on the type of cell to be grown are suitable as the cell culture medium.

Nevertheless, it is also possible to cultivate “suspension cells”. Suspension cells are cells which do not grow as a monolayer or multilayer, i.e. do not adhere to an inert surface. Examples of suspension cells are blood cells, such as leukocytes, or lymphoid cell lines.

Advantages of the present invention consist inter alia in the avoidance of passaging steps during cell expansion and the concomitant greatly simplified automatability of the process and also in the process management which spares the cells.

The advantages of the bioreactor according to the invention will become evident from the following discussion of the use according to the invention of the bioreactor and the methods according to the invention.

The following preferred and/or alternative embodiments can for example be provided for the bioreactor according to the invention:

According to the invention, the bioreactor is preferably disassemblable. According to the invention, the bioreactor can preferably be disassembled into at least three parts.

According to the invention, the bioreactor can preferably be disassembled into at least three parts, namely a) a reactor housing part which surrounds the first reactor region, b) a reactor housing part which surrounds the second reactor region and c) the membrane unit which is liquid-permeable, at least in partial regions.

According to the invention, the bioreactor is preferably modular in its construction.

According to the invention, the bioreactor housing is therefore preferably formed from at least two reactor housing parts, in particular from two reactor housing parts.

Alternatively, the first and the second reactor housing part can also be connected to each other, in particular via a hinge.

Preferably, according to the invention, the first reactor housing part is configured as a reactor cover and the second reactor housing part is configured as a reactor lower part.

According to the invention, the bioreactor preferably contains a reactor lower part, a reactor cover and a membrane unit. According to the invention, the bioreactor preferably consists of a reactor lower part, a reactor cover and a membrane unit. According to the invention, the reactor lower part is preferably upwardly opened. The membrane unit then covers the opening in the reactor lower part in that the membrane of the membrane unit is positioned horizontally on the opening. The reactor cover then has a downwardly directed opening. The reactor cover is then placed onto the reactor lower part, so that the opening in the reactor cover is also covered by the horizontal membrane. The membrane of the membrane unit thus demarcates the reactor region or reactor space formed by the reactor lower part from the reactor region or reactor space formed by the reactor cover.

According to the invention, the reactor space is therefore preferably split up by the membrane unit, which is liquid-permeable in partial regions, into a lower reactor region and into an upper reactor region.

According to the invention, the two reactor housing parts can preferably be connected to each other in an air-tight manner. According to the invention, the two reactor housing parts can be connected to each other in an air-tight manner by a screw connection. However, the person skilled in the art is also familiar with other alternative or additional possibilities for connecting the two reactor housing parts in an air-tight manner, for example by clamping, by clamping devices, by bands, in particular rubber bands, or by ties.

According to the invention, the two reactor housing parts are preferably connected to each other in an air-tight manner during the cultivation of cells in the bioreactor.

According to the invention, the membrane unit preferably consists of at least one membrane and at least one frame. Alternatively, the membrane unit can consist only of at least one membrane.

According to the invention, the membrane unit preferably consists of a membrane and a frame. Alternatively, the membrane unit can consist only of a membrane.

According to the invention, the reactor housing parts preferably have receiving devices, for example slots or protrusions, for receiving and positioning the membrane unit.

In an alternative embodiment the reactor housing parts have receiving devices, for example slots or protrusions, for receiving and positioning the membrane.

In an alternative embodiment the reactor housing parts have receiving devices, for example slots or protrusions, for receiving and positioning the frame of the membrane unit.

If the membrane unit contains a frame, said frame preferably serves to carry and to orient the membrane and to position the membrane in the bioreactor.

The frame, which is optionally provided, can be manufactured simply and inexpensively as a disposable injection-moulded part. The material of the frame, for example plastics material, for example polypropylene or polystyrene, can be selected in such a way that the frame can be sterilised, in particular can be autoclaved. Provision may be made for the frame of the membrane unit to be used several times and the membrane, which is preferably used just once, to be exchangeable in the frame.

However, provision may also be made for the entire membrane unit, including for example the frame, to be configured as a disposable product. The cell culture frame can be manufactured simply and inexpensively as a disposable injection-moulded part and the reactor housing can selectively be reused after sterilisation. This provides a broad spectrum of applications.

In the membrane unit which is preferably provided in accordance with the invention, the membrane is embodied in particular as a membrane insert which is held in the frame, preferably by clamping.

In a particular alternative embodiment according to the invention the membrane area, which serves as the growth area of the cells, can be varied by way of different sized frame inserts, thus allowing the usable cell growth area to be adapted as required. Provision may also be made for the size of the frame to be variable, so that it covers different sized regions of the membrane. This allows the size of the membrane surface available to the cells during the cultivation to be altered, in particular to be increased in size during the growth or propagation of the cells.

The size of the cell growth area formed by the membrane can be increased in different ways without infringing the growth conditions, for example by way of a mechanically variably adjustable cell growth area.

In a further particular alternative embodiment according to the invention the bioreactor can be constructed in such a way that the frame is reversible in the bioreactor, in particular may be reversed in an automated manner, thus allowing the coculture of different cell types, including for example in various media. In this case, according to the invention, provision is then preferably made for both surfaces of the membrane to serve as the growth area for the cells; in particular, one membrane surface can serve as the growth area for a first cell type and the second membrane surface can serve as the growth area for a second cell type. This allows the blood/brain barrier to be modelled, for example.

Provision may also be made for the membrane to be inserted into the bioreactor directly, without a frame.

If the membrane is used without a frame, a tensioning system can be provided for tensioning the membrane in the bioreactor.

Furthermore, during manufacture of the bioreactor, the membrane or the membrane unit can be connected directly thereto, in particular to a reactor housing part, for example to the reactor lower part. This is possible, in particular in disposable bioreactors.

According to the invention, the membrane unit is preferably liquid-permeable through the membrane. According to the invention, the membrane therefore preferably forms the liquid-permeable part, in particular the sole liquid-permeable part of the membrane unit.

In an alternative embodiment according to the invention the membrane can also be replaced by another suitable porous material, for example by a sponge-like material.

According to the invention, the membrane of the membrane unit is preferably disc-shaped or sheet-shaped and is positioned with its two surfaces horizontally between the two reactor regions. Preferably, according to the invention, the entire membrane unit is roughly disc-shaped or sheet-shaped and is positioned with its two surfaces horizontally between the two reactor regions.

According to the invention, the membrane preferably takes up at least 25% of the membrane unit. According to the invention, the membrane preferably takes up at least 50% of the membrane unit. Provision may also be made for the membrane to take up at least 75%, in particular at least 90% of the area of the membrane unit.

According to the invention, the membrane preferably takes up between 25 and 100% of the area of the membrane unit. According to the invention, the membrane preferably takes up between 50% and 100% of the area of the membrane unit.

The membrane serves as the growth area of the cells. According to the invention, only one of the two surfaces of the membrane preferably serves as the growth area of the cells. If the membrane is inserted, in accordance with the invention, preferably horizontally in the bioreactor, according to the invention preferably the upper surface of the membrane serves as the growth area of the cells.

Alternatively, however, in particular in adherent cells, both surfaces of the membrane can also be used as the growth area of the cells.

The person skilled in the art is familiar with suitable liquid-permeable membranes which are suitable for cultivating cells, in particular adherent cells.

The cell growth membrane can for example be made of PET, i.e. polyethylene terephthalate, or a comparable material. The membrane has by definition a porous structure, so that liquids can be exchanged via the membrane.

The cell growth membrane can for example be made of PET, i.e. polyethylene terephthalate, or a comparable material. The use of polycarbonate membranes is, for example, also possible. The membrane has by definition a porous structure, so that liquids can be exchanged via the membrane.

According to the invention, the membrane is preferably selected from the group consisting of PET membranes, PC membranes, nylon membranes, amphoteric nylon membranes, positively charged nylon membranes, negatively charged nylon membranes, PTFE membranes, cellulose ester membranes, cellulose acetate membranes, cellulose nitrate membranes, cellulose mixed ester membranes, regenerated cellulose membranes, Nytran membranes and Nytran SuPerCharge++ membranes.

In an alternative embodiment according to the invention the membrane is a PET membrane. In an alternative embodiment according to the invention the membrane is a PC membrane. In an alternative embodiment according to the invention the membrane is a nylon membrane. In an alternative embodiment according to the invention the membrane is an amphoteric nylon membrane. In an alternative embodiment according to the invention the membrane is a positively charged nylon membrane. In an alternative embodiment according to the invention the membrane is a negatively charged nylon membrane. In an alternative embodiment according to the invention the membrane is a PTFE membrane. In an alternative embodiment according to the invention the membrane is a cellulose ester membrane. In an alternative embodiment according to the invention the membrane is a cellulose acetate membrane. In an alternative embodiment according to the invention the membrane is a cellulose nitrate membrane. In an alternative embodiment according to the invention the membrane is a cellulose mixed ester membrane. In an alternative embodiment according to the invention the membrane is a regenerated cellulose membrane. In an alternative embodiment according to the invention the membrane is a Nytran+ membrane. In an alternative embodiment according to the invention the membrane is a Nytran SuPerCharge++ membrane.

A coating of the cell growth membrane or application of a surface structure is also selectively possible.

According to the invention, the membrane preferably has a pore size of at least 0.01 μm. According to the invention, the membrane preferably has a pore size of at least 0.1 μm. According to the invention, the membrane preferably has a pore size of at least 0.35 μm.

According to the invention, the membrane preferably has a pore size of at most 20 μm. According to the invention, the membrane preferably has a pore size of at most 10 μm.

According to the invention, the membrane preferably has a pore size of at least 0.01 μm, in particular 0.1 μm and at most 20 μm, in particular at most 10 μm.

According to the invention, the membrane preferably has a pore size of at least 0.35 μm, in particular 0.4 μm and at most 9 μm, in particular at most 8 μm.

A pore size of 0.4 μm, 0.45 μm, 1.0 μm, 1.2 μm, 3.0 μm, 5.0 μm or 8.0 μm can for example be provided.

The person skilled in the art is familiar with suitable pore sizes of membranes ensuring both a sufficient passage of liquid, in particular a passage of cell culture medium, and at the same time a good cultivation of the cells.

According to the invention, the membrane of the membrane unit preferably has a pore density of at least 10⁵ pores per cm². According to the invention, the membrane of the membrane unit preferably has a pore density of at most 10⁸ pores per cm².

According to the invention, the membrane of the membrane unit preferably has a pore density of at least 10⁵ pores per cm² and at most 10⁸ pores per cm². A pore density of 0.1×10⁶, 0.2×10⁶, 0.4×10⁶, 2×10⁶, 22×10⁶ or 100×10⁶ pores per cm² can for example be provided.

The person skilled in the art is familiar with suitable pore densities of membranes ensuring both a sufficient passage of liquid, in particular a passage of cell culture medium, and at the same time a good cultivation of the cells.

Provision may for example also be made for the membrane to have an area expanse of from 50 cm² to 120 cm², in particular from 75 cm² to 85 cm² In particular, the membrane can have an area expanse of about 80 cm².

According to the invention, each of the two reactor regions has an opening suitable for letting in and/or letting out a liquid.

The openings connect the interior of the bioreactor to the exterior of the bioreactor; they are therefore openings which pass through the bioreactor housing.

According to the invention, each of the two reactor housing parts preferably has an opening suitable for letting in and/or letting out a liquid.

The openings can preferably be in the form of connections. The bioreactor can be connected to other devices, for example in an automated overall system for producing tissue from cell cultures, through the connections, for example via hoses, and a liquid, in particular a cell culture medium or a solution for detaching the cells, can be supplied to the bioreactor or removed from the bioreactor through the connections.

The openings can also be in the form of septa.

The openings can also be in the form of pipetting openings.

According to the invention, the first reactor region preferably has an opening suitable for letting in a liquid. According to the invention, the first reactor region preferably has an opening suitable for letting in and letting out a liquid.

According to the invention, the openings are preferably closable. Alternatively, the openings can also be configured so as to be non-closable, for example when the bioreactor is connected by the openings to an overall system via hoses.

According to the invention, the opening, which is suitable for letting in and/or letting out a liquid, in the first reactor region is preferably positioned at the side or on the upper side of the reactor housing part. According to the invention, the opening, which is suitable for letting in and/or letting out a liquid, in the first reactor region is positioned at the side of the reactor housing part.

According to the invention, the second reactor region preferably has an opening suitable for letting in and letting out a liquid.

According to the invention, the opening, which is suitable for letting in and/or letting out a liquid, in the second reactor region is preferably positioned at the side or on the upper side of the reactor housing part. According to the invention, the opening, which is suitable for letting in and/or letting out a liquid, in the second reactor region is positioned at the side of the reactor housing part.

According to the invention, the openings suitable for letting in and/or letting out a liquid are designed as valves. However, they can also be designed as closable or non-closable nozzles or connections to which a feed line or a discharge line, for example in the form of a hose, can be attached. This allows continuous or step-by-step supplying of fresh liquid, while removing old liquid at the same time or beforehand. This is advantageous, in particular when the bioreactor according to the invention is used in an automated device for the cultivation of cells.

Alternatively, however, the liquid can also be supplied and/or removed differently, in particular in a manual use of the bioreactor according to the invention, for example by adding the liquid by pipette or pouring it in or by removing the liquid by pipette, draining it out, or pouring it out.

According to the invention, the liquid is preferably a cell culture medium or a solution for detaching the cells from the membrane.

According to the invention, the first and/or the second reactor region can be configured in such a way that they are formed by a reactor chamber, the actual reactor region, and by a guide system connecting the openings to the reactor chambers.

According to the invention, provision may alternatively be made for the reactor regions or a reactor region to be formed only by reactor chambers or a reactor chamber.

According to the invention, provision may alternatively be made for the bioreactor to be reversible.

According to the invention, the bioreactor preferably has at least one ventilation opening.

According to the invention, the first reactor housing part preferably has at least one ventilation opening. According to the invention, the first reactor housing part, in particular the reactor cover, preferably has a ventilation opening.

According to the invention, the at least one ventilation opening is preferably provided with a sterile filter.

Provision may also be made for the ventilation opening to be closable.

According to the invention, the bioreactor preferably has at least one pipetting opening.

According to the invention, the first reactor housing part preferably has at least one pipetting opening. According to the invention, the first reactor housing part preferably has a pipetting opening.

According to the invention, provision may be made for the bioreactor to have two pipetting openings. In this case, provision may be made for one pipetting opening to form an access to the first reactor region and the second pipetting opening to form an access to the second reactor region. In this case, the second pipetting opening can for example be located on the upper side of the reactor cover and be in the form of a line to the lower, second reactor region. The line can for example be guided alongside the membrane or the membrane unit. For example, specially shaped webs can allow liquid to be fed into the lower reactor region via the pipetting opening.

According to the invention, provision may in particular be made for the two pipetting openings to be positioned on the upper side of the reactor cover.

Alternatively, provision may also be made for the pipetting openings to be the two openings suitable for letting in and/or letting out a liquid. In this embodiment provision is therefore made for the pipetting openings to be present not in addition to, but instead of the two openings suitable for letting in and/or letting out a liquid.

In particular, provision may be made for the bioreactor to contain, as the sole openings, the two pipetting openings and a ventilation opening.

According to the invention, the pipetting opening is preferably closable, in particular closable in an air-tight manner. The pipetting opening can if appropriate also be provided with a liquid-permeable sterile filter.

Alternatively, it is also possible to provide further, in particular closable, openings allowing the addition and/or removal of liquid, in particular cell culture medium or additives, into the bioreactor or out of the bioreactor.

According to the invention, the first reactor housing part preferably has at least one ventilation opening, in particular provided with a sterile filter, and/or a closable pipetting opening.

In an alternative embodiment the bioreactor is suitable for receiving metrological devices, for example electrodes; in this case, plug-in slots or the like are therefore provided for receiving the metrological devices.

According to the invention, metrological devices, which allow the measuring of data, for example for measuring TEER values, are preferably integrated into the bioreactor.

By measuring the TEER value (TEER=transepithelial electrical resistance), the density of the cell population can be determined. Thus, it is possible to determine the point in time at which the cells on the membrane have reached the desired cell density, in particular the point in time at which the cell layer has become almost confluent or confluent.

The term “confluence” is used to describe the closest possible arrangement of adherent cells at the surface of a culture vessel surface, i.e., in the present case, the membrane surface. The confluence differs from cell line to cell line.

Shortly before or when confluence is reached as a result of the growing and/or the propagation of the cells, the cells can be harvested or passaged.

According to the invention, the bioreactor preferably has devices, in particular electrodes, for measuring data.

According to the invention, the bioreactor preferably has devices, in particular electrodes, for measuring TEER values.

Alternatively, only plug-in slots can also be provided for the devices, in particular electrodes for measuring TEER values.

According to the invention, the first reactor housing part preferably has at least one electrode, in particular one electrode, for measuring TEER values. According to the invention, the second reactor housing part preferably has at least one electrode, in particular one electrode, for measuring TEER values.

Preferably, according to the invention, the first reactor region has an electrode for measuring TEER values and the second reactor region also has an electrode for measuring TEER values.

As a result of the optionally integrated electrodes, the population density of the cells on the carrier structure can be monitored in an preferred manner by means of measuring the TEER value.

According to the invention, metrological devices, which allow the measurement of the opacity, the pH, the glucose content and/or the oxygen content of the liquid, in particular of the cell culture medium, are preferably integrated into the bioreactor. A visual opacity measurement can for example be provided.

According to the invention, the first reactor region preferably has a measuring means for measuring the amount of liquid or the volume of liquid. According to the invention, the second reactor region preferably has a measuring means for measuring the amount of liquid or the volume of liquid. By measuring the amount of liquid in the bioreactor or in partial regions of the bioreactor, it is for example possible to pour in exactly the desired amount of enzyme solution for detaching the cells in order to be able to carry out in a simple manner the method, which is preferred hereinafter in accordance with the invention, for detaching the cells, in particular to be able to carry it out automatically.

However, provision may also be made for the amount of liquid in the bioreactor not to have to be measured, as the volumes of the reactor regions are precisely known, and the desired or required amounts are as a result likewise known and can be added, for example in a computer-controlled manner, with precise metering.

In particular, the second reactor region, in particular the reactor region surrounded by the reactor lower part, can have a volume of from 5 ml to 20 ml.

In particular, the second reactor region, in particular the reactor region surrounded by the reactor lower part, can have a volume of at least 10 ml. In particular, the second reactor region, in particular the reactor region surrounded by the reactor lower part, can have a volume of at most 15 ml.

According to the invention, the two reactor regions of the bioreactor according to the invention are preferably roughly the same size and/or take up roughly the same volume.

According to the invention, the upper reaction region is in this case preferably somewhat larger, in particular larger than the lower reaction region.

According to the invention, the bioreactor preferably has support elements for fixing the membrane. According to the invention, the first reactor housing part preferably has support elements for fixing the membrane. According to the invention, the second reactor housing part preferably has support elements for fixing the membrane. According to the invention, the first and second reactor housing parts preferably have support elements for fixing the membrane. According to the invention, the reactor lower part and the reactor cover preferably have support elements for fixing the membrane.

According to the invention, the bioreactor housing, in particular the reactor housing parts, is made of plastics material or plexiglass. The person skilled in the art is familiar with suitable plastics materials, but also other possible materials for the bioreactor housing. Possible plastics materials are for example polypropylene or polystyrene.

According to the invention, the bioreactor housing, in particular the reactor housing parts, is preferably transparent, at least in partial regions. It is in particular possible to provide transparent partial regions allowing the cells on the membrane to be observed.

According to the invention, the bioreactor housing, in particular the reactor housing parts, can preferably be sterilised, in particular be autoclaved. The housing of the bioreactor can thus be used several times. This provides a broad spectrum of applications.

However, provision may also be made to design the bioreactor as a disposable product.

In particular, a bioreactor can be provided as a disposable product having one, several or all the following optional features:

All the components of the bioreactor are configured as a disposable product.

The reactor housing of the bioreactor is made of plastics material, in particular polypropylene or polystyrene.

The reactor housing is manufactured by injection moulding.

The membrane unit or the membrane is fastened to the reactor lower part.

The bioreactor cover can be raised from the unit made up of the reactor lower part and membrane unit.

The bioreactor has, as the sole openings, two pipetting openings and selectively a ventilation opening.

The two pipetting openings are located on the upper side of the reactor cover.

The first pipetting opening leads to the first reactor region and the second pipetting opening leads to the second reactor region.

The bioreactor has septa.

The bioreactor has a width of at least 6 cm and at most 12 cm, in particular between 8 cm and 9 cm, and a length of at least 10 cm and at most 14 cm, in particular of at least 12 cm and at most 13 cm.

The bioreactor has a width and a length in the dimensions of a standardised multiwell plate.

The bioreactor has a height of at least 1 cm and at most 10 cm.

The membrane has an area expanse of from 50 cm² to 120 cm², in particular from 75 cm² to 85 cm². In particular, the membrane can have an area expanse of about 80 cm².

Of course, a bioreactor of this type can also have other features and further features of the present invention.

A bioreactor according to the invention can be configured in a GMP-compliant manner, and the risk of contamination or of the loss of sterility during the cultivation of the cell cultures is reduced. In this case, the use of disposable elements is reduced to a minimum without significantly increasing the risk of contamination.

The person skilled in the art is familiar with possible shapes of the bioreactor. The person skilled in the art can readily adapt the shape of the bioreactor to his requirements. According to the invention, the bioreactor is preferably box-shaped, carton-shaped or can-shaped.

In particular, the bioreactor can have a length and a width corresponding to standardised lengths and widths of cell culture vessels, for example multiwell plates.

According to the invention, a bioreactor which does not itself contain a micro-pump is preferred.

The present invention also relates to the use of the bioreactor according to the invention.

According to the invention, the use of a bioreactor according to the invention for cultivating cells is preferred. According to the invention, the use of a bioreactor according to the invention for cultivating and expanding cells is preferred.

According to the invention, the cells are preferably adherent cells.

According to the invention, the cells are preferably eukaryotic cells. According to the invention, the cells are preferably animal cells. According to the invention, the cells are preferably mammalian cells, in particular human, bovine or murine cells.

Alternatively, however, it is also possible to cultivate prokaryotic cells, plant cells or fungal cells, in particular if they are adherent cells.

According to the invention, the use of a bioreactor according to the invention in an automated device for producing tissue from cell cultures is preferred.

Alternatively, however, the bioreactor according to the invention can also be used manually, for example be assembled under a clean bench and be filled and then be incubated in an incubator.

The bioreactor can selectively be used as a stand-alone module or as a component in an overall system, for example for producing cell tissue from biopsates.

The present invention also relates to methods for the cultivation of cells, a bioreactor according to the invention being used for the cultivation. According to the invention, the cells are preferably cultivated and expanded in the method.

The bioreactor according to the invention allows the carrying-out of a fully automated method for the cultivation of cells that can be carried out preferably under GMP-compliant conditions.

The present invention also relates in particular to a method for the cultivation of cells, containing the steps a) providing a bioreactor according to the invention, b) applying cells to the membrane of the membrane unit, c) cultivating the cells in the bioreactor.

Provision is in this case made for the steps to be carried out in the specified order. According to the invention, steps a), b) and c) are preferably carried out immediately in succession, without further intermediate steps.

According to the invention, the cells are preferably cultivated in step b) in a liquid, in particular in a cell culture medium.

According to the invention, the cells are preferably adherent cells. According to the invention, the cells are preferably eukaryotic cells. According to the invention, the cells are preferably animal cells. According to the invention, the cells are preferably mammalian cells, in particular human, bovine or murine cells.

According to the invention, the cells are preferably left in step b) to adhere; then, at the beginning of step c), the bioreactor is flooded with cell culture medium for the first time. This takes place preferably via one of the openings in the bioreactor.

In order to achieve the meeting of growth conditions, for example in relation to cell/cell contacts, and the multiple passaging necessitated thereby, a novel method was developed for populating the cell growth membrane with primary cells. According to the invention, said method is preferably used in step b).

According to the invention, the membrane provided as the cell growth area is in this case preferably not wetted in its entirety with cell liquid during populating; instead, individual cell clusters, distributed uniformly on the cell growth area, are produced.

According to the invention, the cells suspended in a cell culture medium are in step b) preferably pipetted dropwise onto the membrane at roughly constant, in particular at constant spacing, using a pipette.

In a membrane having a length of from 10 cm to 14 cm and a width of from 6 cm to 10 cm, the cells are in particular applied at a total volume of liquid of from 1 ml to 10 ml, in particular from 3 ml to 5 ml. The total amount of liquid is split up into drops.

During this process, the following parameters are variable for a person skilled in the art: drop volume, drop spacing, cell concentration in the drop and/or waiting time until the first exchange of media.

These parameters also allow the cultivation behaviour of the respective cells to be altered.

The cell concentration within a drop leads in this case to maintenance of the cell/cell contacts necessary for cell growth while at the same time increasing the size of the available cell growth area to a multiple of the cell growth area of a standard laboratory culture vessel. In addition, this preferred populating method ensures a uniform distribution of the cells over the porous material as the culturing area, whereas in standard culture vessels the cells are distributed randomly and the distribution is often higher in the edge regions than at the centre. A uniform cell distribution has the advantage that the growth conditions on the populated porous material are unitary. As a result of the now sufficiently large cell growth area, in particular additional passaging steps of the cells, in particular in the automated process, can be avoided, thus eliminating the need for a large number of handling steps which otherwise impede the automated process and damage the cells.

According to the invention, the drop volume is preferably between at least 2.5 μl and at most 30 μl. The drop volume can for example be 2.5 μl, 5 μl, 10 μl, 20 μl or 30 μl.

According to the invention, preferably between 10 and 500, in particular between 25 and 350 cells, for example about 20, 40, 80, 160 or 320 cells, are present in a drop during seeding.

According to the invention, the cells are preferably applied to the porous surface at a cell concentration during seeding of from approx. 4,000 to approx. 20,000 cells/cm², in particular from approx. 4,000 to approx. 16,000 cells/cm². For example, 4,000 cells/cm², 5,000 cells/cm², 8,000 cells/cm², 10,000 cells/cm², 16,000 cells/cm² or 20,000 cells/cm² can be applied to the porous material, in particular the membrane. For a membrane area of approx. 80 cm², that would be roughly 320,000-1,280,000 cells for each bioreactor.

A uniform dropwise populating of the cell growth area allows the size of the cell growth area to be increased to a multiple without infringing the conditions for successful cell expansion. The size of cell growth area that is necessary for passaging-free cell expansion is in this way achieved.

The additional damaging of the cells during passaging as a result of the action of enzyme reactions or mechanical loads can also be reduced to a minimum, in particular to a single detachment of the cells at the end of the cultivation period.

The moment of the cell harvest after cultivation has been concluded thus preferably remains as the single necessary detaching process of the cells. Furthermore, the harmful effect of the enzyme on the cells can be minimised by a new detaching method.

Alternatively, the cell growth membrane can also be populated in a manner other than the pipetting of droplets, for example by spraying with cells suspended in liquid.

According to the invention, adherent cells are preferably left in step b) to adhere to the membrane and then further cultivated while adding liquid, in particular cell culture medium.

According to the invention, the liquid is preferably renewed in step c). This can be carried out in particular through the openings suitable for letting in and/or letting out a liquid. The liquid can be renewed continuously, semicontinuously or step-by-step.

Preferably, according to the invention, new liquid is in this case introduced through the opening in the first or the second reactor housing part and old liquid is removed through the opening in the first or the second reactor housing part.

Preferably, according to the invention, new liquid is in this case introduced through the opening in the reactor cover and old liquid is removed through the opening in the reactor lower part.

Alternatively, however, provision may also be made to introduce and to remove the liquid through pipetting openings.

Alternatively, however, provision may also be made not to renew the liquid.

Provision may be made to metrologically test the state of the cells for a broad range of parameters via a process control of step c), for example via electrodes for measuring the TEER value and/or the automated supply of media. In particular, provision may be made to determine the duration of step c) by measuring the TEER value. In this case, step c) can preferably be ended when the desired cell density is reached, in particular when the cells are almost confluent or are confluent.

According to the invention, the cells are preferably detached from the membrane surface in a step d) after the cultivating. According to the invention, the detaching is preferably carried out using a solution suitable for detaching the cells. According to the invention, the detaching preferably takes place enzymatically.

The enzymatic detaching can be carried out using enzyme-containing solutions such as are also used in the prior art for detaching adherent cells. In particular, the person skilled in the art will use trypsin, in particular in an EDTA solution, as the enzyme. The person skilled in the art is in this case familiar with suitable trypsin concentrations.

According to the invention, the enzyme is preferably trypsin. However, use may also be made of other enzymes known to the person skilled in the art, such as for example Accutase or rProtease.

The enzyme solution can be poured in either through one of the openings in the bioreactor or through the opened bioreactor.

According to the invention, the solution suitable for detaching the cells is preferably poured into the bioreactor, and thus brought to the membrane, through one of the openings suitable for letting in and/or letting out a liquid.

The design according to the invention of the bioreactor and the position of the openings suitable for letting in and/or letting out a liquid allow the carrying-out of a gentle detaching process and a rise in the vitality of the detached cells.

According to the invention, the detachment of the cells adhered to the membrane is preferably controlled via an enzyme reaction, wherein the enzyme can act merely through the porous membrane at the contact of the cell layer with the membrane.

According to the invention, the cells are preferably detached from the membrane surface after the cultivating, wherein a solution which is suitable for detaching the cells is poured into the reactor region bordering the surface of the membrane to which no cells are applied through the opening in this reactor region, so that the solution has contact with the membrane. According to the invention, the solution suitable for detaching the cells preferably has contact only to the cell branches of the cell that are connected to the membrane.

According to the invention, the solution suitable for detaching the cells is preferably introduced into the bioreactor through the opening in the reactor lower part. According to the invention, only so much of the solution is preferably introduced that the filling level of the solution just reaches the membrane. As the cells preferably adhere to the membrane surface facing the reactor cover, the solution suitable for detaching the cells thus has contact only to the cell branches of the cells that are connected to the membrane.

Likewise, it is possible, by varying the design of the reactor, to modify the detaching process and to assist the enzymatic detachment by, or if appropriate to replace it with, physical methods. Physical methods include an application of pressure to the reactor lower part; furthermore, it is also possible to assist the detaching process by knocking and/or shaking the bioreactor. Physical methods also include the use of ultra-sound or of rising air bubbles from the underside of the reactor.

In an alternative embodiment according to the invention, in step d), the cells are enzymatically detached, the detaching being assisted by physical methods. The detaching process is thus accelerated.

Provision may be made for the cells also to be detached by excess pressure in step d) or after step d). For example, the solution suitable for detaching the cells can be pressed through the membrane with pressure.

For example, provision may be made, in step d), to remove the cell culture medium, in particular via the guide system, and to fill both reactor regions with PBS/EDTA and to incubate them for 1 to 30 min. This process can be repeated 1 to 2 times, depending on the cell type. Both reactor regions can then be completely emptied and the lower reactor region can subsequently be filled with an enzyme solution. For a defined range of from 0.2 to 10 min and by application of a defined pressure in the range of from 50 to 2,000 Pa, the enzyme solution can be passed through the pores, having a diameter in the range of from 0.1 to 10 μm, of the membrane, even to the points of contact of the cells to the upper side of the membrane. Subsequently, an incubation step having a duration in the range of from 0.5 to 10 min can be carried out.

In order to assist the detaching process, a pressure in the same pressure range can be applied during the detaching or subsequently, i.e. after the detaching, in the lower chamber. Said pressure can additionally be modulated with a frequency of from 0.1 to 200 Hz. In addition, the process can be intensified by way of mechanical excitation of the entire reactor. The pressures which are applied produce, in particular, volume flows through the membrane in the range of from 0.5 to 20 ml/(min cm²).

Provision may be made for, after completion of step d), the cells to be removed from the membrane in a step e). For example, the cells can be taken up in cell culture medium, in particular in serum-containing cell culture medium, and then suction-extracted or removed by pipette.

Provision may also be made, after completion of step d), to introduce in step e) more of the solution suitable for detaching the cells and to thereby push the cells away from the porous surface.

Provision may be made for the cells to be singled out in step d) or after step d), for example with the aid of trypsin.

According to the invention, the method is preferably carried out in an automated manner, in particular in an automated device for producing tissue from cell cultures and/or using a robot.

According to the invention, the method is preferably carried out in an automated device for producing tissue from cell cultures.

The bioreactor according to the invention allows in a preferred embodiment an automated carrying-out of the cell cultivation of cells, in particular of adherent cells.

In this case, an advantageous increase in the size of the cell growth area is possible while meeting the cell growth conditions.

The bioreactor according to the invention also allows an avoidance of passaging steps which, in particular in an automated process, are very complex and can easily damage the cells.

The bioreactor according to the invention preferably provides an integration of metrology, in particular for measuring TEER values. The harvest moment can thus be determined precisely, including in particular in automated processes.

The bioreactor according to the invention allows a method according to the invention for detaching cells from the membrane that spares the cells and in which only the contact points of the cells to the membrane enter into contact with the enzyme used for detaching.

The bioreactor according to the invention, its use according to the invention and the methods according to the invention can be utilised in particular in automated systems which are operated for the cultivation of adherent cells.

However, the bioreactor according to the invention, its use according to the invention and the methods according to the invention can also be utilised on a laboratory scale, in particular in order to minimise the risk of contamination and to avoid a direct metering of liquids to the preparation.

The bioreactor according to the invention, its use according to the invention and the methods according to the invention can be utilised in particular in the cultivation of cells for tissue engineering.

The bioreactor according to the invention, its use according to the invention and the methods according to the invention can be utilised in particular in the cultivation of various cell types for producing artificial skin as an in-vitro test system or as a transplant.

The bioreactor according to the invention, its use according to the invention and the methods according to the invention can be utilised in particular also in the cultivation of various cell types for producing artificial cartilage transplants.

Embodiments, which are preferred and alternative in accordance with the invention, of the bioreactor according to the invention are also to be understood as being embodiments, which are preferred and alternative in accordance with the invention, of the uses of the bioreactor and as being embodiments, which are preferred and alternative in accordance with the invention, of the methods according to the invention.

Embodiments, which are preferred and alternative in accordance with the invention, of the uses according to the invention are also to be understood as being embodiments, which are preferred and alternative in accordance with the invention, of the bioreactor according to the invention and as being embodiments, which are preferred and alternative in accordance with the invention, of the methods according to the invention.

Embodiments, which are preferred and alternative in accordance with the invention, of the methods according to the invention are also to be understood as being embodiments, which are preferred and alternative in accordance with the invention, of the uses of the bioreactor and as being embodiments, which are preferred and alternative in accordance with the invention, of the bioreactor according to the invention.

Particular embodiments of the invention are disposed in the dependent claims.

Further advantages of the invention will emerge from the figures described hereinafter, in which:

### BRIEF DESCRIPTION OF THE DRAWINGS

FIG. 1 shows, in an illustration which is not true-to-scale, an embodiment, which is preferred in accordance with the invention, of the bioreactor according to the invention in the assembled state;

FIG. 2 shows, in an illustration which is not true-to-scale, an embodiment, which is preferred in accordance with the invention, of the bioreactor according to the invention in the dismantled state;

FIG. 3 shows various views of an alternative reactor cover according to the invention;

FIG. 4 shows various views of an alternative reactor lower part according to the invention; and

FIG. 5 illustrates a method, which is preferred in accordance with the invention, for applying the cells to the membrane of the membrane unit of the reactor.

### DETAIL DESCRIPTION

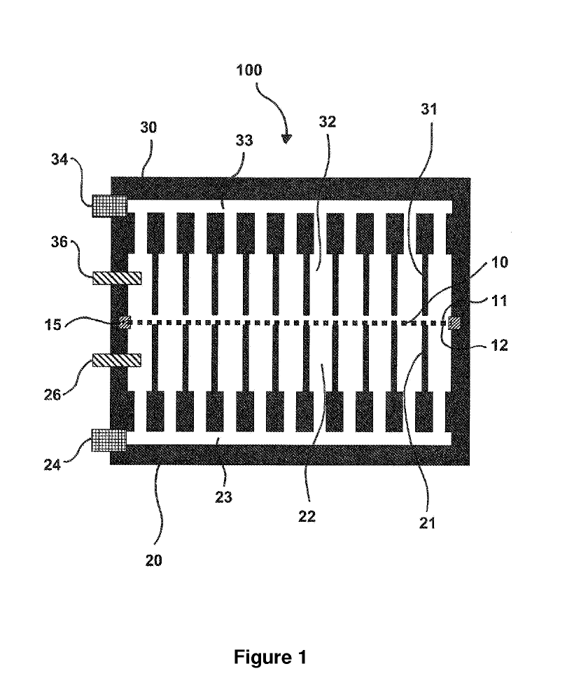

FIG. 1 shows, in an illustration which is not true-to-scale, an embodiment, which is preferred in accordance with the invention, of the bioreactor (100) according to the invention in the assembled state. The bioreactor consists of a reactor lower part (20), a reactor upper part (30) and a membrane unit formed from a membrane (10) and a frame (15). The reactor lower part (20) and reactor upper part (30) have support elements (21, 31) which fix the membrane (10). The reactor lower part (20) and reactor upper part (30) are connected in an air-tight manner, so that only the openings (24, 34), which are in the form of connections, connect the reactor chambers (22, 32) to the environment of the bioreactor (100) via the guide systems (23, 33). The guide systems (23, 33) ensure a continuous and uniform supply of the cell growth membrane over the entire area. An optional ventilation opening or pipetting opening is not shown. The reactor lower part (20) and reactor upper part (30) both have electrodes (26, 36) for measuring the TEER value of cultivated cells. The growth process can be monitored and the optimum harvest moment determined via the TEER values which are measured.

The membrane (10) has an upper side (11) and an underside (12). Preferably, adherent cells are cultivated on the upper side (11) of the membrane. In this case, the reactor chambers (22, 32) are flooded with cell culture medium via one of the openings (24, 34). When the TEER value measurement via the electrodes (26, 36) reveals that an optimum cell density has been reached, for example that the cells have grown confluent, the cell culture medium can be drained out via the lower opening (24). A trypsin-containing solution can then be introduced into the bioreactor (100) through the opening (24). So much of the solution is introduced that the solution just obtains contact to the membrane (10). As a result, the cells are detached from the membrane surface (11); however, only the membrane contact points of the cells enter into contact with the trypsin, so that the cells are damaged less than during a conventional detaching of the cells.

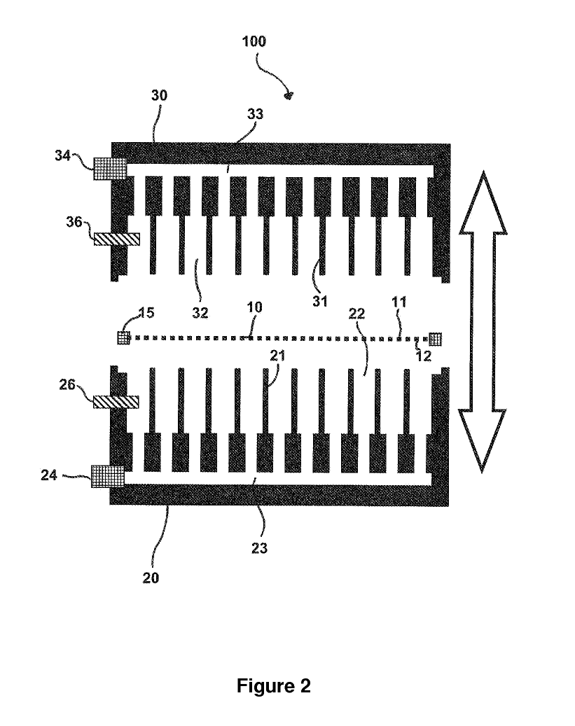

FIG. 2 shows, in an illustration which is not true-to-scale, the bioreactor (100), which is preferred in accordance with the invention, from FIG. 1 in the dismantled state. The reactor upper part (30) and the membrane unit, which is formed from a membrane (10) and a frame (15), are separated from the reactor lower part (20). The membrane surface (11) of the membrane (10) is thus accessible and can be populated with cells, for example by pipetting-on the cells. Afterwards, the reactor upper part (30), the membrane unit and the reactor lower part (20) can be reassembled.

Shown, again, are openings (24, 34), reactor chambers (22, 32) and guide systems (23, 33), as well as electrodes (26, 36).

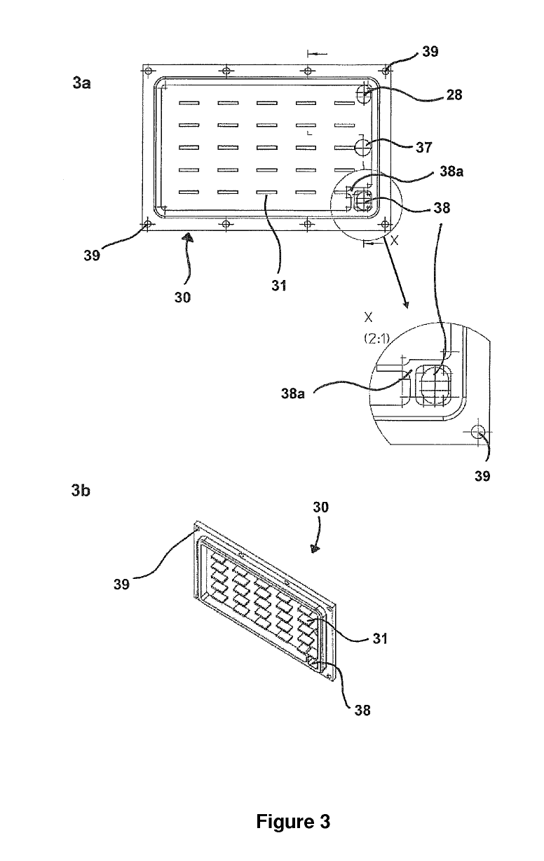

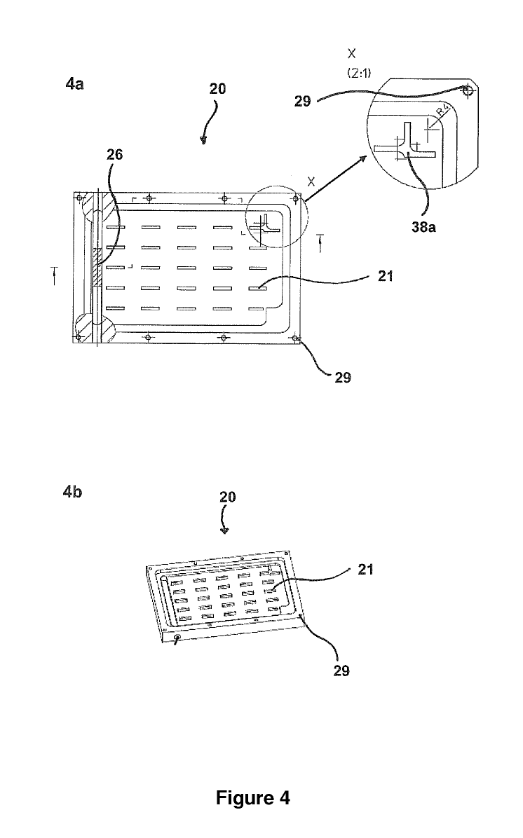

FIG. 3 and FIG. 4 show various views of a reactor cover (30) which is preferred in accordance with the invention and a reactor lower part (20) which is preferred in accordance with the invention. The reactor cover (30) and reactor lower part (20) have roughly the length and the width of a multiwell plate. For both reactor housing parts, the support elements (21, 31) may be seen.

FIG. 3 also shows a ventilation opening (37) and two pipetting openings (28, 38). The pipetting opening (28) serves as an access to the upper reaction chamber. The pipetting opening (38) serves as an access to the lower reaction chamber. This access is made possible by way of a guide system which bypasses the membrane, which is positioned between the reaction chambers, in a sealed manner.

The enlarged view shown in FIGS. 3a and 4a shows a specific embodiment of a web (38a) which facilitates the introduction of liquid through the pipetting opening.

FIG. 4 shows a mounted electrode (26). The two reactor housing parts (20, 30) can be screwed to each other through the boreholes (29, 39).

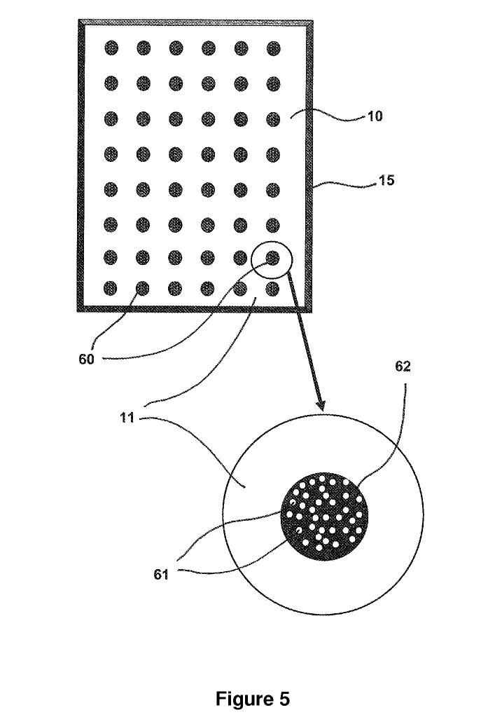

FIG. 5 illustrates a method, which is preferred in accordance with the invention, for applying the cells to the membrane of the membrane unit of the reactor. A membrane unit with a membrane (10) and a frame (15) is shown. The membrane upper side (11) of the membrane (10) is uniformly covered with cell-containing drops (60) from cell culture medium. The enlarged view shows a detail of the membrane surface (11) with a drop (61) and cells (62) contained therein.

Table 1 shows by way of example various membranes which can be used:

TABLE 1
Exemplary listing of membranes which can be used
in the bioreactor according to the invention

| | | | Pore size | Pore density/ |
|---|---|---|---|---|
| Type | Company | Material | (μm) | cm² |
| M2020 | Oxyphen | PC | 0.4 | 100 × 10⁶ |
| M2014 | Oxyphen | PC | 5.0 | 0.4 × 10⁶ |
| M2017 | Oxyphen | PC | 8.0 | 0.1 × 10⁶ |
| M2019 | Oxyphen | PET | 0.4 | 100 × 10⁶ |
| M2016 | Oxyphen | PET | 1.0 | 22 × 10⁶ |
| M2015 | Oxyphen | PET | 3.0 | 2 × 10⁶ |
| M2018 | Oxyphen | PET | 8.0 | 0.2 × 10⁶ |
| IDO,45 | Pall | nylon | 0.45 | |
| ID1,2 | Pall | nylon | 1.2 | |
| ID3,0 | Pall | nylon | 3.0 | |
| ID5,0 | Pall | nylon | 5.0 | |
| BDA | Pall | amphoteric nylon | 0.45 | |
| BDB | Pall | positively charged | 0.45 | |
| | | nylon | | |
| BDC | Pall | negatively charged | 0.45 | |
| | | nylon | | |
| MC CM | Millipore | PTFE | 0.4 | |
| MC HA | Millipore | cellulose ester | 0.45 | |
| MC PCF | Millipore | PC | 0.4 | |
| OE67 | Whatman | cellulose acetate | 0.45 | |
| CN45 | Whatman | cellulose nitrate | 0.45 | |
| ME25 | Whatman | cellulose mixed ester | 0.45 | |
| RC55 | Whatman | regenerated cellulose | 0.45 | |
| BA85 | Whatman | pure cellulose | 0.45 | |
| N45 | Whatman | Nytran+ | 0.45 | |
| SuPer45 | Whatman | Nytran SuPer- | 0.45 | |
| | | Charge+++ | | |

The method therefore makes provision to pipette the primary cells suspended in a cell culture medium dropwise onto the cell growth membrane at constant spacing, using a pipette.

Only once the cells have adhered is the bioreactor (100) assembled and flooded with cell culture medium. The cell concentration within the drop leads in this case to maintenance of the cell/cell contacts necessary for cell growth while at the same time increasing the size of the available cell growth area to a multiple of the cell growth area of a standard laboratory culture vessel. In addition, this populating method ensures a uniform distribution of the cells over the culturing area, whereas in standard culture vessels the cells are distributed randomly and the distribution is often higher in the edge regions than at the centre. A uniform cell distribution has the advantage that the growth conditions within the culture vessel are unitary. As a result of the now sufficiently large cell growth area, a passaging of the cells in the automated process can be dispensed with, thus eliminating the need for a large number of handling steps which otherwise impede the automated process.

Additional damaging of the cells during multiple passaging as a result of the action of enzyme reactions or mechanical stress can also be reduced to a minimum, i.e. a single detachment of the cells at the end of the cultivation period, as the membrane offers sufficient space for growth and for propagation of the cells.

The point in time of the cell harvest after cultivation has been concluded thus remains as the single necessary detaching process of the cells. Furthermore, the harmful effect of the enzyme on the cells can be minimised by the preferred detaching method.

1. A bioreactor for the cultivation of cells, wherein reactor space of the bioreactor is subdivided by a membrane unit, which is liquid-permeable at least in partial regions, into two reactor regions and wherein each of these two reactor regions has an opening suitable for letting in and/or letting out a liquid.

2. The bioreactor according to claim 1, wherein the bioreactor can be disassembled into at least three parts, namely
a) a first reactor housing part which surrounds a first reactor region;
b) a second reactor housing part which surrounds a second reactor region; and
c) a membrane unit which is liquid-permeable, at least in partial regions.

3. The bioreactor according to claim 2, wherein the first reactor housing part is forms a reactor cover and the second reactor housing part is forms a reactor lower part.

4. The bioreactor according to claim 2, wherein the first reactor housing part and second reactor housing part can be connected to each other in an air-tight manner, in particular by a screw connection.

5. The bioreactor according to claim 1, wherein the membrane unit consists only of a membrane.

6. The bioreactor according to claim 1, wherein the membrane unit is disc-shaped and is positioned with its two surfaces horizontally between the two reactor regions.

7. The bioreactor according to claim 1, wherein the membrane unit comprises a membrane which takes up between 25% and 100% of the area of the membrane unit.

8. The bioreactor according to claim 1, wherein the membrane unit comprises a membrane which has a pore size of at least 0.1 μm and at most 20 μm.

9. The bioreactor according to claim 1, wherein the membrane unit has a membrane with a pore density of at least 10⁵ pores per cm² and at most 10⁷ pores per cm².

10. The bioreactor according to claim 2, wherein the first reactor housing part has at least one ventilation opening, in particular provided with at least one of a sterile filter and a closable pipetting opening.

11. The bioreactor according to claim 1, wherein the bioreactor has electrodes for measuring TEER values.

12. The bioreactor according to claim 1, wherein the bioreactor is box-shaped or can-shaped.

13. A method for using a bioreactor, the method comprising using a bioreactor having reactor space which is subdivided by a membrane unit, which is liquid-permeable at least in partial regions, into two reactor regions and wherein each of these two reactor regions has an opening suitable for letting in and/or letting out a liquid for cultivating cells.

14. The method of using a bioreactor, wherein the method comprises using the bioreactor method comprising using a bioreactor having reactor space which is subdivided by a membrane unit, which is liquid-permeable at least in partial regions, into two reactor regions and wherein each of these two reactor regions has an opening suitable for letting in and/or letting out a liquid in an automated device to produce tissue from cell cultures.

15. A method for the cultivation of cells including
providing a bioreactor having a reactor space subdivided into two reactor regions by a membrane unit having, which is liquid-permeable at least in partial regions, and wherein each of these two reactor regions has an opening suitable for letting in and/or letting out a liquid;
applying cells to the membrane of the membrane unit; and
cultivating the cells in the bioreactor.

16. The method according to claim 15, wherein after the cultivation of the cells, further detaching the cells from the membrane surface, by a solution which is suitable for detaching the cells is poured into the reactor region bordering a surface of the membrane to which no cells are applied through an opening of this reactor region, so that the solution has contact with the membrane.

17. The method according to claim 16, wherein the method is carried out in an automated manner, in particular in an automated device for producing tissue from cell cultures and/or using a robot.

18. The method according to claim 15, wherein the method is carried out in an automated manner, in particular in an automated device for producing tissue from cell cultures and/or using a robot.
